(19) United States
(12) Patent Application Publication
(10) Pub. No.: US 2002/0032388 A1
(43) Pub. Date: Mar. 14, 2002

(54) Disposable sensor for measuring respiration and method of forming the same

(76) Inventors: Helgi Kristbjarnarson, Reykjavik (IS); Kormakur Hermannson, Reykjavik (IS); Eggert Gudjonsson, Reykjavik (IS)

Correspondence Address: PILLSBURY WINTHROP LLP, 1600 TYSONS BOULEVARD, MCLEAN, VA, 22102, US

(21) Appl. No.: 09/785,553

(22) Filed: Feb. 20, 2001

Related U.S. Application Data
(63) Continuation-in-part of application No. 09/661,156, filed on Sep. 13, 2000.

(51) Int. Cl. A61B 5/08

(52) U.S. Cl. 600/538

(57) ABSTRACT

The present invention is directed to a disposable sensor for monitoring and measuring the respiration of a patient. The disposable sensor includes at least one flexible woven ribbon adapted to encircle a portion (e.g., the chest or abdomen) of the patient. The woven flexible ribbon includes a plurality of elastic threads extending in a lengthwise direction. The elastic threads are interconnected by an inelastic thread extending in a widthwise direction. Each flexible ribbon includes a conductor strip secured thereto. The conductor strip extends in a zig-zag or other predetermined pattern. The disposable sensor also includes a connector assembly for connecting and securing a first free end of the ribbon to a second free end of the ribbon.

### CROSS REFERENCE TO RELATED APPLICATIONS

This application is a continuation-in-part of U.S. patent application Ser. No. 09/661,156, filed Sep. 13, 2000, entitled “DISPOSABLE SENSOR FOR MEASURING RESPIRATION”.

### FIELD OF THE INVENTION

The present invention relates to a disposable sensor for use in measuring respiration of a patient. In particular, the present invention relates to a method of forming a respiratory inductive plethysmograph (“RIP”) disposable sensor formed from a flexible stretchable ribbon having a conductor secured thereto. The disposable sensor is cut to size by a health care provider sized to encircle a patient to measure respiration.

### BACKGROUND OF THE INVENTION

Respiratory inductive plethysmograph monitoring apparatus are used to measure and monitor the respiration of a patient. Typically, the RIP monitoring apparatus includes a conductive loop closely encircling a body member. The inductance of the conductive loop is a measure of the cross sectional area encircled. Changes in inductance reflect the respiration of the patient. The conductive loop is connected to an electronic monitoring device, which includes circuitry that reliably and accurately measures changes in the inductance of the conductive loop mounted on the body. Respiratory movements of the wearer result in changes in the cross-sectional areas and hence in the inductance of the conductive loop. Once these changes in inductance are converted to an electrical signal for the conductive loop, the signal is calibrated by the electronic monitoring device to accurately measure the volume of respiration. To ensure accurate monitoring of the patient's respiration, it is important that the conductor loop fit snugly about the patient's torso such that the expansions and contractions of the conductor closely follow the expansions and contractions of the chest and abdomen. Further, since it is impractical to render the conductors expandable, some other mechanism for accommodating expansion and contraction of the conductors must be employed.

U.S. Pat. No. 4,308,872 to Watson et al entitled “Method and Apparatus for Monitoring Respiration” discloses an apparatus for monitoring respiration. In one embodiment, the monitoring apparatus includes a tubular stretch bandage in the form of a long sleeveless sweater worn closely fitted over the torso of a patient. A conductor is attached to the sweater in a number of turns around the torso from the lower abdomen to the upper chest, and so will provide a measure of area averaged over the entire torso. More turns may be placed over one portion of the torso and fewer over other portions, if it is desired to give greater weight to changes in area of one portion of the torso relative to others. The multi-turn loop is closed by a vertical section returning to the starting point. Both ends of the loop are electrically connected to an electronic circuit module, which is located on the patient's lower side. In another embodiment, the monitoring apparatus includes two elastic tubes located about the upper chest and the lower abdomen of the patient. Conductors are mounted in a single turn loop circumferentially of tubes. Snap fasteners are provided for holding the band together.

U.S. Pat. No. 4,807,640 to Watson et al., entitled “Stretchable Band-Type Transducer Particularly Suited For Respiration Monitoring Apparatus” discloses a monitoring apparatus having a conductor, which is supported on a strip of woven fabric securable about a patient's torso. The fabric strip is stitched under tension by a plurality of longitudinally extending elastic stitches such that when the tension in the strip is released, the fabric becomes bunched or puckered along its entire length. An insulated wire conductor is stitched to one side of the fabric in a zigzag pattern. The stretching of the fabric in a longitudinal direction is accommodated by the puckers or folds with corresponding extension of the wire being accommodated by a widening and flattening of the saw tooth pattern. In use, the length of the band in its unstretched condition should be less than the circumference of the encircled portion of the torso of the patient such that the band may be stretched for a snug fit. To accommodate connection of the wire to the monitoring apparatus, the conductor is secured to the fabric such that both ends of the conductor terminate at the same longitudinal edge of the band. The ends of the conductor are soldered to connecting pins which are then secured in shrink tubing such that the tips of the connecting pins are exposed. The shrink tubing is stapled to the ends of the band. The conductors are then secured to a monitoring device.

The '640 patent also discloses a RIP monitoring apparatus having a stretchable band. The stretchable band includes a piece of nonwoven fabric, and a piece of tissue paper secured to the nonwoven fabric. A piece of elastic material is adhesively secured between the nonwoven fabric and the tissue paper along a substantial portion of the length. The elastic material in a stretched condition when the first and second pieces of material are in a flat condition. As described above, the nonwoven fabric and the tissue paper define crosswise puckers when the elastic material is in an unstretched condition for accommodating stretching of the band when the elastic material is stretched. A conductor is adhesively secured between the nonwoven fabric and the tissue paper. The stretchable band has a rather complex construction, which requires multiple distinct manufacturing steps. A fastener, such as, for example, a hook and loop fastener, is secured to the opposing ends of the band for securing the band to the patient. Free ends of the conductor extend from the ends of the band are connected to a LC oscillator circuit contained a housing secured to one end of the band. The LC oscillator circuit is then electrically connected to a monitoring apparatus.

None of these RIP monitoring apparatus, however, are suited for mass production. Each of these devices has a rather complex construction, which results in higher manufacturing costs and final product costs. Due to the expense, it is impractical to discard them after a single use. Yet in the medical field, where sanitary considerations are paramount for sound medical reasons as well as psychological ones, it is preferred to render disposable any apparatus that comes into contact with the patient, such as occurs with the RIP bands. Furthermore, these RIP monitoring apparatus are cut in predetermined lengths and can not be easily sized to fit individual users. Hospitals would need to maintain a substantial inventory of different sized RIP bands.

### OBJECTS OF THE INVENTION

It is accordingly an object of the present invention to provide a RIP band sensor device and method for forming the same which overcome the aforementioned drawbacks.

It is therefore an object of the present invention to provide a low cost sensor device for monitoring a patient's respiration that can be discarded after a single use.

It is another object of the present invention to provide a low cost sensor device that can be woven.

It is another object of the present invention to provide a low cost RIP ribbon sensor device that can be readily and easily sized to an individual.

It is another object of the present invention to provide a disposable sensor for monitoring the volume of an expandable organ from which the respiration of the user, for example, may be determined.

It is a further object of the present invention to provide a RIP sensor device that can easily be connected to a monitoring apparatus.

It is yet a further object of the present invention to provide a flexible RIP ribbon band that can be mass produced.

It is yet another object of the present invention to form a woven RIP sensor ribbon having elastomeric threads extending in a lengthwise direction.

It is another object of the present invention to provide a RIP sensor ribbon having a conductor strip woven into the band.

It is another object of the present invention to provide a connector assembly for a disposable sensor that establishes an electrical connections with a conductor wire in the disposable sensor.

Additional objects and advantages of the invention are set forth, in part, in the description which follows, and, in part, will be apparent to one of ordinary skill in the art from the description and/or practice of the invention.

### SUMMARY OF THE INVENTION

In response to the foregoing challenges, applicants have developed an innovative disposable sensor for monitoring and measuring the respiration of a patient. The disposable sensor includes at least one woven flexible ribbon adapted to encircle a portion (e.g., the chest or abdomen) of the patient. A conductor strip is woven into the ribbon. It is contemplated that the conductor strips extends in a zig-zag or other predetermined pattern on the ribbon.

In accordance with the present invention, the disposable sensor monitors changes in volume of an expandable organ of a patient. The sensor includes at least one flexible ribbon adapted to encircle a portion of the patient. Each flexible ribbon includes a plurality of expandable threads extending along a longitudinal axis of the ribbon and at least one non-elastic thread traversing the plurality of elastic threads in a woven pattern to interconnect the plurality of elastic threads. Each expandable thread includes an inner elastic strand, and an outer cover strand surrounding the elastic strand. The plurality of elastic threads and the at least one non-elastic thread form a woven flexible ribbon.

In accordance with the present invention, a conductor is secured to the woven flexible ribbon. The conductor may be secured to the woven flexible ribbon in a zig-zag pattern.

Each expandable thread includes an inner elastic strand, and an outer cover strand surrounding the elastic strand.

The disposable sensor also includes a connector assembly for connecting and securing a first free end of the ribbon to a second free end of the ribbon. The connector assembly is operatively coupled to the conductor strip, and is further adapted to be connected to a monitoring device. In operation, changes in inductance of the conductor strip are transmitted through the connector assembly to the monitoring device. The connector assembly for the disposable sensor includes a compression assembly for mechanically compressing the conductor strip. It is contemplated that the conductor strip may include a conductive wire having an outer insulation layer. The compression assembly cuts away the outer insulation layer.

It is also contemplated that the connector assembly of the disposable sensor may include a first connector portion for releasably receiving the first free end of the flexible ribbon. The first connector portion is adapted to engage a portion of the conductor strip located adjacent the first free end. The connector assembly also includes a second connector portion for releasably receiving the second free end of the flexible band. The second connector portion being adapted to engage a portion of the conductor strip located adjacent the second free end.

The present invention is also directed to a method of measuring and monitoring changes in volume of an expandable organ of a patient. By measuring the changes in volume, the respiration of a patient may be measured and monitored. The method includes providing a supply of a flexible disposable sensor ribbon, cutting a length of the flexible disposable sensor ribbon to encircle a torso of the patient, securing a first end of the length to a releasable connector assembly, securing a second end of the length the releasable connector assembly, connecting the releasable connector assembly to a monitoring assembly, and monitoring changes in inductance of the flexible disposable sensor ribbon to measure and monitor the changes in volume of an expandable organ of the patient.

The method further includes disposing of the length of the flexible disposable sensor ribbon after monitoring changes in inductance of the flexible disposable sensor band.

### BRIEF DESCRIPTION OF THE DRAWINGS

The invention will be described in conjunction with the following drawings in which like reference numerals designate like elements and wherein:

FIG. 1 is a view of the RIP sensor ribbon band in accordance with the present invention located on a patient;

FIG. 2 is a schematic view of the RIP sensor ribbon of FIG. 1 in accordance with a preferred embodiment of the present invention;

FIG. 3 is an enlarged schematic view of the RIP sensor ribbon of FIG. 2.

FIG. 4 is a schematic view illustrating the connector assembly and RIP sensor ribbon in accordance with the present invention;

FIG. 5 is a schematic view illustrating a preferred connector assembly and RIP sensor ribbon in accordance with the present invention;

FIG. 6 is an enlarged schematic view of the connector assembly of FIG. 4;

FIG. 7 is a partial schematic view of the male and female connector assemblies of the connector assembly of FIG. 4 in an open disengaged position;

FIG. 8 is a partial schematic view of the male and female connector assemblies of the connector assembly of FIG. 4 in a closed engaged position; and

FIG. 9 is a cross-sectional view of the lengthwise thread of the ribbon sensor.

### DETAILED DESCRIPTION OF THE PREFERRED EMBODIMENTS

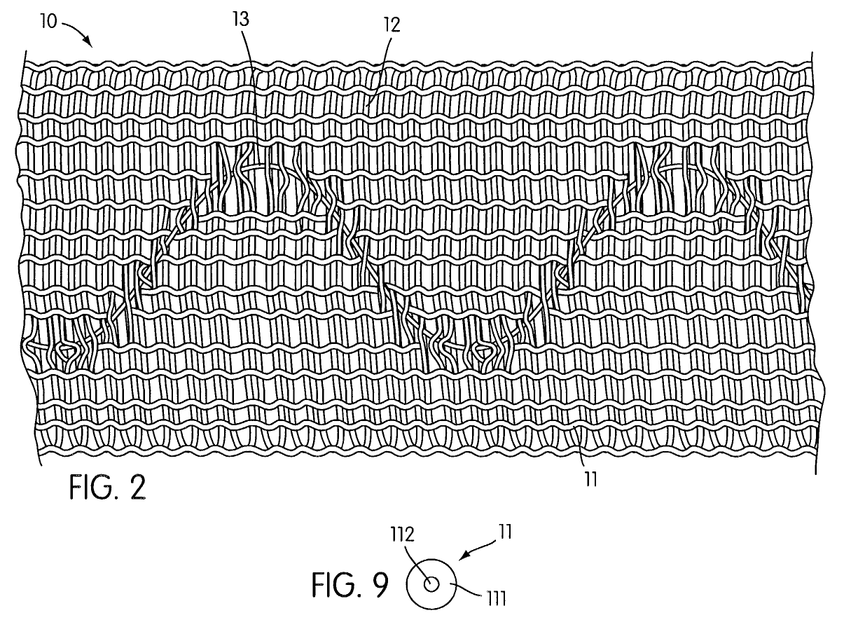

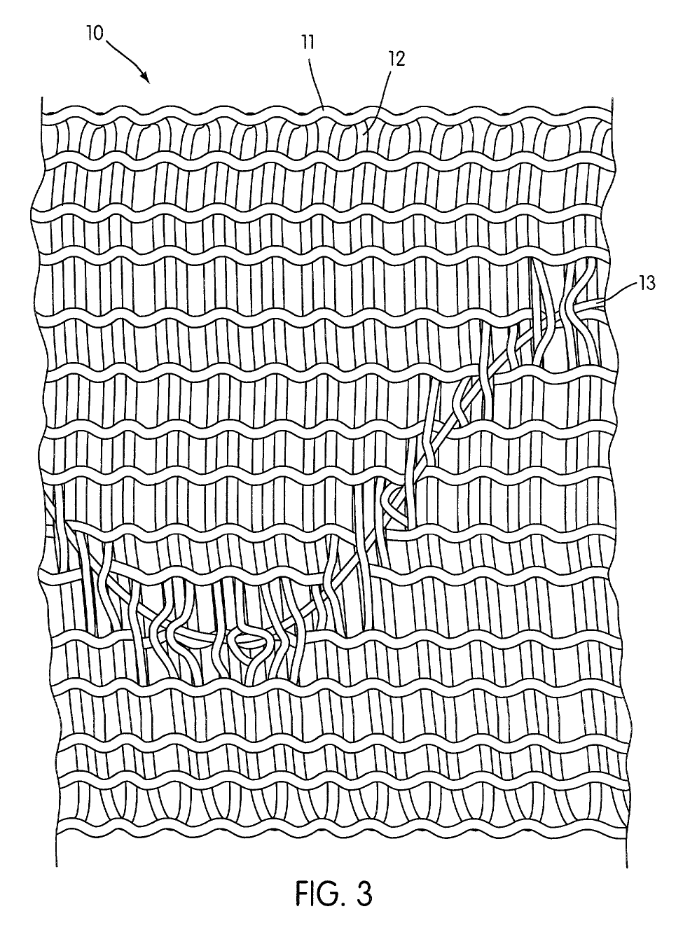

The flexible disposable RIP sensor ribbon in accordance with the present invention will now be described in detail. The RIP sensor ribbon 10 includes a single strip of flexible stretchable ribbon that can be easily cut to size for a particular patient from a supply of ribbon 10. The sensor ribbon 10 includes a plurality of expandable threads or strands 11 extending in the lengthwise direction, as shown in FIGS. 2 and 3. A thread or strand 12 traverses the elastic threads 11 in a widthwise direction to interconnect the threads 11 to form the ribbon 10. The sensor ribbon 10 further includes a conductor strip 13. It is preferred that the conductor strip 13 be disposed in a zig-zag pattern, as shown in FIGS. 2 and 3, which permits flexing of the ribbon 10 as the patient breaths to monitor and measure volume changes in an expandable organ and thus the respiration of the patient without damaging the conductor strip 13. While the zig zag pattern is preferred from a manufacturing and flexibility standpoint, other layouts of the conductor strip 13 are contemplated to be within the scope of the present invention provided the layout permits expansion and contraction of the ribbon 10.

The flexible ribbon 10 preferably has a woven construction, as shown in FIGS. 2 and 3. The threads or strands 11 are flexible and elastic to permit expansion of the ribbon 10 in response to changes in volume of the expandable organ. It is preferable that the threads 11 are formed from a nylon thread 111 knitted or formed around an elastic thread 112, as shown in FIG. 9. The present invention, however, is not limited to the use of nylon thread for forming the threads 11; rather, other materials having similar properties are considered to be well within the scope of the present invention. Furthermore, the elastomeric thread 112 and nylon thread 111 can be replaced with a single elastomeric strand. In the preferred embodiment, the ribbon 10 includes fifteen expandable threads 11. The present invention, however, is not limited to fifteen threads; rather, it is contemplated additional threads may be used to increase the width of the ribbon 10. Additionally, additional threads may be used to increase the diversity of the ribbon 10. In the alternative, fewer threads may be used to decrease the width of the ribbon 10.

A single lateral strand 12 is woven between the strands 11, as shown in FIGS. 2 and 3. The lateral strand 12 is preferably formed of a flexible material that is not elastic. The lateral strand 12 extends in a first direction across and interconnecting the threads 11 then in a second direction opposite the first direction. This pattern is repeated to form the ribbon 10. The strands 11 and 12 form a loosely woven flexible ribbon. It is preferable that the thread 12 is formed from nylon. As shown in FIGS. 2 and 3, the thread 12 includes a plurality of individual strands. The present invention, however, is not limited to the use of nylon thread; rather, other synthetic materials are contemplated. Furthermore, organic materials such as cotton may be used so long as the resulting threads are substantially inelastic.

The conductor strip 13 is then woven into the ribbon 10 by threading the strip 13 between threads 11 and 12. It is preferable that the conductor strip 13 extends through the individual strands of the thread 12 such that the conductor strip 13 is secured to the ribbon 10. With this arrangement, the conductor strip 13 is made integral with the ribbon 10. As shown in FIGS. 2 and 3, the conductor strip 13 is spaced from the edge of the ribbon 10. It, however, is contemplated that the conductor strip 13 may extend substantially to the edge of the ribbon 10.

In the alternative, it is contemplated that the conductor strip 13 may be aligned on the plurality of elastic threads 11. The strip 13 and expandable threads 11 are then interconnected by thread 12. It is also contemplated that a suitable adhesive may be used to secure the strip 13 to the ribbon 10.

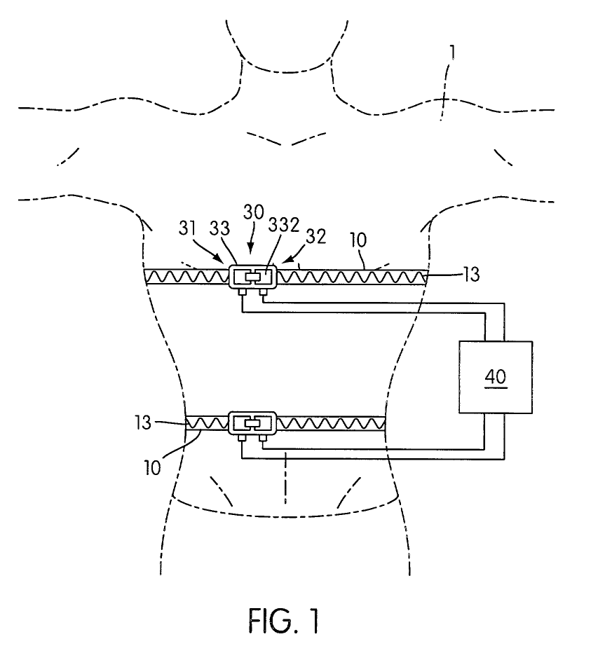

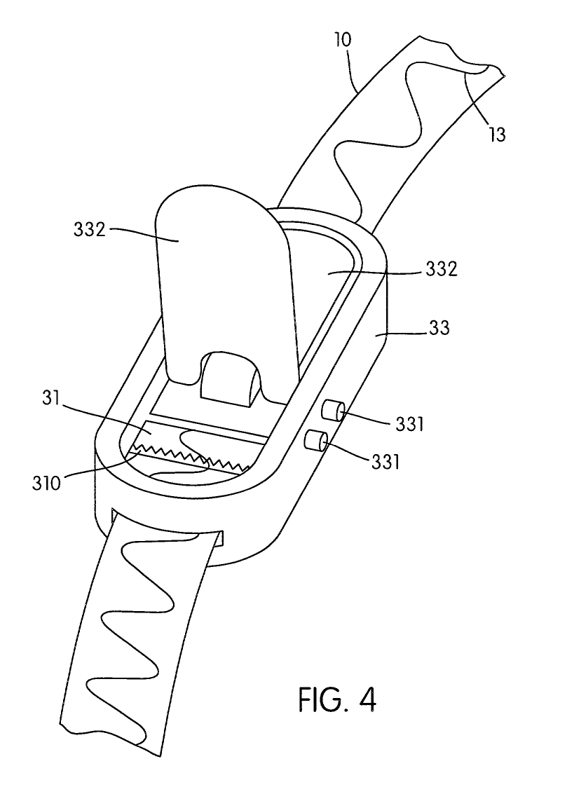

The operation and use of the RIP sensor ribbon 10 connected to a connector assembly 30 to measure and monitor the respiration of a patient will now be described. The technician or health care provider dispenses a sufficient length of ribbon 10 from the dispensing box or roll to encircle the abdomen of the patient 1, as shown in FIG. 1. The length of ribbon 10 is then cut. A first end of the ribbon 10 is secured to a first connector portion 31 of a connector assembly 30, as shown in FIG. 4. The first connector portion 31 includes a piercing assembly 310 for piercing and engaging the ribbon 10. The piercing assembly 310 also makes contact with the connector 13 within the ribbon 10. A second end of the ribbon 10 is secured to a second connector portion 32 of the connector assembly 30. Like the first connector assembly 31, the second connector portion 32 includes a piercing assembly for piercing and engaging the ribbon 10. The piercing assembly also makes contact with the connector 13 within the ribbon 10.

The ribbon 10 is then stretched around the torso of the patient. The first and second connector assemblies 31 and 32 are then secured to the housing 33 of the connector assembly 30. The housing 33 includes connection outputs 331. A monitoring apparatus 40 is electrically connected to the connection outputs 331, as shown in FIG. 1. The housing 33 further includes a latching mechanism 332 for securing the connector assemblies 31 and 32 within the housing 33. After the ribbon 10 is secured to the patient and the monitoring apparatus 40 is connected to the connection outputs 331, changes in inductance in the conductor strip 13 corresponding to the respiration of the patient can be measured and monitored. These changes in inductance are converted to an electrical signal for the conductive loop, the signal is calibrated by the electronic monitoring device to accurately measure the volume of respiration. The monitoring device 40 can then monitor and record the respiration of the patient. The use of the sensor ribbon 10 is not limited to patients; rather, it is contemplated that the sensor ribbon 10 may be used to measure the respiration of a person while exercising.

It is contemplated that more than one ribbon 10 may be used to encircle the chest and abdomen of the patient, as shown in FIG. 1. A separate connector assembly 30 is used for each ribbon 10. The separate ribbons 10 may be connected to a single monitoring device 40. Furthermore, the ribbons 10 are not limited to placement around the user's abdomen; rather, it is contemplated that the ribbons may be located around a portion of the arm or leg of the user.

After the monitoring operation is complete, the ribbon 10 can be removed from the patient. The ribbon 10 can be disconnected from the connector assembly 30 and discarded. It is contemplated that the connector assembly 30 may be disposed of after use or reused if desired. The present invention permits the sizing of the sensor 10 to be patient specific. Unlike the prior art, it is not necessary for a hospital to maintain a large supply of different sized sensors for different patients.

The operation and use of the RIP sensor ribbon 10 connected to a preferred connector assembly 50 to measure and monitor the respiration of a patient will now be described. Like the embodiment described above, the technician or health care provider dispenses a sufficient length of ribbon 10 from the dispensing box or roll to encircle the abdomen of the patient 1, as shown in FIG. 1. The length of ribbon 10 is then cut. A first end of the ribbon 10 is secured to a female connector portion 51 of a connector assembly 50, as shown in FIG. 4. A second end of the ribbon 10 is secured to a male connector portion 52 of the connector assembly 50.

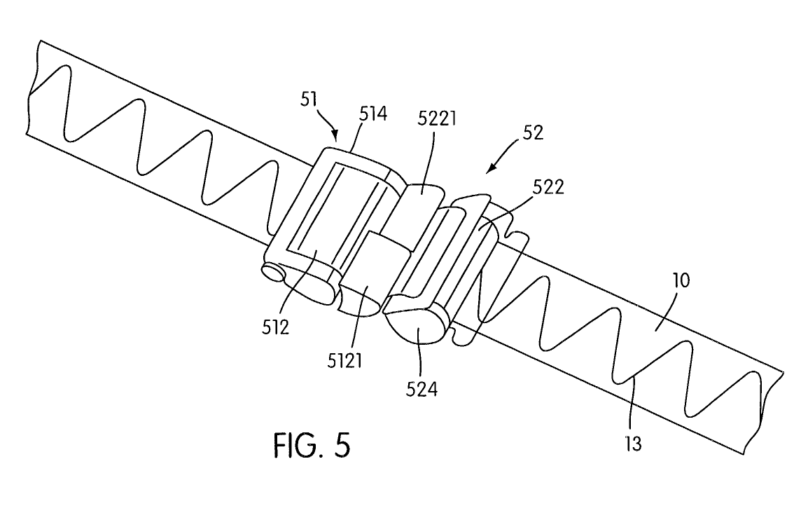

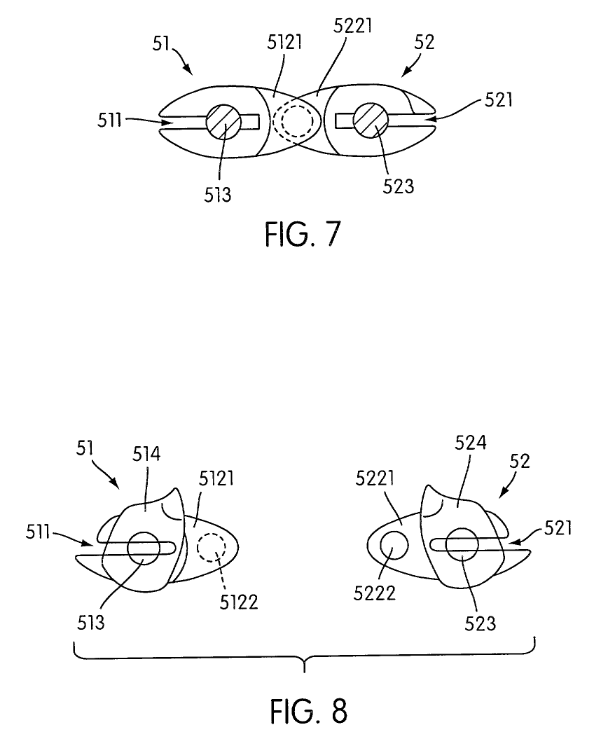

The ribbon 10 is then stretched around the torso of the patient. The female and male connector assemblies 51 and 52 are then engaged, as shown in FIGS. 5 and 7. The connector assembly 50 is then secured to the monitoring apparatus 40. After the ribbon 10 is secured to the patient and the monitoring apparatus 40, changes in inductance in the conductor strip 13 corresponding to volume changes in an expandable organ during respiration of the patient can be measured and monitored. These changes in inductance are converted to an electrical signal for the conductive loop, the signal is calibrated by the electronic monitoring device to accurately measure the volume of respiration. The monitoring device 40 can then monitor and record the respiration of the patient.

The first end of the ribbon 10 is secured to the female connector portion 51 by inserting the ribbon 10 into the channel 511, shown in FIG. 8. The channel 511 extends through a stationary housing portion 512 and a rotatable member 513. The rotatable member 513 is secured to a rotatable actuator assembly 514. The rotatable actuator assembly 514 rotates between an open position, shown in FIG. 8, and a closed position, shown in FIG. 7.

When in the closed position, shown in FIG. 7, the ribbon 10 and, in particular, the conductor strip 13 is compressed between the rotatable actuator assembly 514 and the stationary housing portion 512. With this arrangement, any protective coating on the conductor strip 13 is stripped as the actuator assembly 514 rotates to the closed position. The compression of the conductor strip 13 establishes an electrical connection between the strip 13 and the female connector assembly 51. The female connector assembly 51 can then be connected to the monitoring apparatus 40 in a similar manner as described above in connection with connector assembly 30.

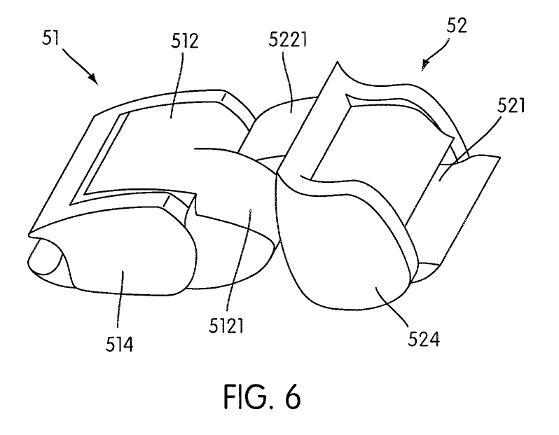

The second end of the ribbon 10 is secured to the male connector portion 52 by inserting the ribbon 10 into the channel 521, shown in FIG. 8. The channel 521 extends through a stationary housing portion 522 and a rotatable member 523. The rotatable member 523 is secured to a rotatable actuator assembly 524. The rotatable actuator assembly 524 rotates between an open position, shown in FIG. 8, and a closed position, shown in FIG. 6.

Like the female connector assembly 51, the conductor strip 13 is compressed between the rotatable actuator assembly 524 and the stationary housing portion 522. With this arrangement, any protective coating on the conductor strip 13 is stripped as the actuator assembly 524 rotates to the closed position. The compression of the conductor strip 13 establishes an electrical connection between the strip 13 and the male connector assembly 52. The male connector assembly 52 can then be connected to the monitoring apparatus 40 in a similar manner as described above in connection with connector assembly 30.

The female connector assembly 51 and male connector assembly 52 can be releasably secured together in the following manner. The stationary housing portion 512 includes an engagement portion 5121 having an engagement recess 5122 formed therein, as shown in FIG. 5. The recess 5122 is adapted to releasably receive an engagement projection 5222 on engagement portion 5221 of the male connector assembly 52. It is contemplated that other suitable means may be used to secure the connector assemblies 51 and 52 together.

It is contemplated that more than one ribbon 10 may be used to encircle the chest and abdomen of the patient, as shown in FIG. 1. A separate connector assembly 30 or 50 may used for each ribbon 10. The separate ribbons 10 may be connected to a single monitoring device 40.

After the monitoring operation is complete, the ribbon 10 can be removed from the patient. The ribbon 10 can be easily disconnected from either connector assembly 30 or 50 and discarded. It is contemplated that the connector assembly 30 or 50 may be disposed of after use or reused if desired. The present invention permits the sizing of the sensor 10 to be patient specific. Unlike the prior art, it is not necessary for a hospital to maintain a large supply of different sized sensors for different patients.

It will be apparent to those skilled in the art that various modifications and variations may be made without departing from the scope of the present invention. Thus, it is intended that the present invention covers the modifications and variations of the invention, provided they come within the scope of the appended claims and their equivalents.

What is claimed is:

1. A disposable sensor for monitoring changes in volume of an expandable organ of a patient, comprising:
at least one flexible ribbon adapted to encircle a portion of the patient, wherein each flexible ribbon comprises:
a plurality of elastic threads extending along a longitudinal axis of the ribbon;
at least one non-elastic thread traversing the plurality of elastic threads in a woven pattern to interconnect the plurality of elastic threads, wherein the plurality of elastic threads and the at least one non-elastic thread form a woven flexible ribbon; and
a conductor secured to the woven flexible ribbon.

2. The disposable sensor according to claim 1, wherein the conductor is secured to the woven flexible ribbon in a zig-zag pattern.

3. The disposable sensor according to claim 1, wherein each of the plurality of elastic threads comprises:
an inner elastic strand; and
an outer cover strand surround the elastic strand.

4. The disposable sensor according to claim 1, wherein the conductor is secured to the at least one non-elastic thread.

5. The disposable sensor according to claim 4, wherein the conductor is secured to the woven flexible ribbon in a zig-zag pattern.

6. A method of forming a disposable 'sensor ribbon, comprising:
providing a plurality of expandable threads, wherein the plurality of expandable threads extend substantially parallel to a longitudinal axis of the ribbon;
providing at least one thread for interconnecting the plurality of expandable threads;
interconnecting the at least one thread and the plurality of expandable threads to form a flexible ribbon; and
securing a conductor strip to the ribbon.

7. The method according to claim 6, wherein interconnecting the at least one thread and the plurality of expandable threads includes weaving the at least one thread to the plurality of expandable threads.

8. The method according to claim 6, wherein securing the conductor strip to the ribbon includes securing the ribbon in a zig-zag pattern.

9. The method according to claim 6, wherein securing the conductor strip to the ribbon includes securing the ribbon to the at least one thread.
